(19) United States
(12) Patent Application Publication
(10) Pub. No.: US 2022/0370464 A1
(43) Pub. Date: Nov. 24, 2022

(54) COMPOSITIONS AND METHODS OF TREATING OR PREVENTING OCULAR INFECTIONS WITH FILOCICLOVIR

(71) Applicant: Microbiotix, Inc., Worcester, MA (US)

(72) Inventors: Islam Hussein, Marlboro, MA (US); Terry L. Bowlin, Maineville, OH (US); Richard Gauthier, Montreal (CA)

(21) Appl. No.: 17/622,613

(22) PCT Filed: Jun. 24, 2020
(86) PCT No.: PCT/US20/39258

§ 371 Date: Dec. 23, 2021

Related U.S. Application Data
(60) Provisional application No. 62/866,006, filed on Jun. 25, 2019.

(51) Int. Cl. A61K 31/522; A61K 9/00; A61K 47/44; A61K 47/10; A61K 9/08; A61P 31/12

(52) U.S. Cl. CPC A61K 31/522; A61K 9/0048; A61K 47/44; A61K 47/10; A61K 9/08; A61P 31/12

(57) ABSTRACT

The present invention is related to therapeutics and prophylactics for the treatment and/or prevention of ocular viral infections in humans and other mammals. Disclosed are methods of treating or preventing ocular viral infections in mammals, in particular caused by adenoviral infections, by administration of an effective amount of filociclovir (FCV).

### CROSS REFERENCE TO PRIORITY APPLICATIONS

This application claims priority to U.S. Provisional Appln. No. 62/866,006 filed Jun. 25, 2019, the contents of which are incorporated herein in their entirety.

### STATEMENT REGARDING FEDERALLY SPONSORED RESEARCH

This invention was made with government support under grant R44 AI054135 awarded by the National Institutes of Health. The United States Government has certain rights in the invention.

### FIELD OF THE INVENTION

This invention is in the field of therapeutic drugs to treat or prevent ocular infections and diseases of the eye. In particular, the present invention is directed to the use of filociclovir (FCV) in the treatment or prevention of ocular infections in mammals, and in particular the treatment or prevention of adenoviral ocular infections in humans. Filociclovir may be advantageously used alone or in combination with anti-inflammatory agents, and/or antibacterial agents, and/or immunomodulatory agents, and/or antiviral agents.

### BACKGROUND OF THE INVENTION

With over 50 known serotypes, adenoviruses (AdVs) are ubiquitous viruses that infect various mucoepithelial cells of the body, including the mucosa of the eye. Other primary targets of AdV include the genitourinary tract, respiratory tract, and gastrointestinal tract (Gonçalves et al., “Adenovirus: from foe to friend,” Rev. Med. Virol. 16(3): 167-186 (2006)). Acute conjunctivitis is a common condition, estimated to affect 6 million people each year in the United States (Udeh et al., “Cost effectiveness of a point-of-care test for adenoviral conjunctivitis,” Am. J. Med. Sci., 336(3): 254-264 (2008)). AdV is the most common cause of infectious conjunctivitis, affecting people of all ages and demographics, and accounts for up to 75% of all conjunctivitis cases (Jhanji et al., “Adenoviral keratoconjunctivitis,” Surv. Ophthalmol., 60(5): 435-443 (2015)). Adenoviral conjunctivitis can cause significant discomfort and lost productivity. Although mostly self-limiting, in some cases it can lead to complications from long-term immune-mediated sequelae (Ford et al., “Epidemiology of epidemic keratoconjunctivitis,” Epidemiol. Rev., 9: 244-261 (1987)).

Outbreaks of epidemic keratoconjunctivitis are usually caused by AdV types 8, 19, 37 and 54, whereas pharyngoconjunctival fever outbreak is due mostly to AdV types 3, 4, and 7 (Chigbu et al., ‘Pathogenesis and management of adenoviral keratoconjunctivitis,” Infection and Drug Resistance, 11: 981-993 (2018)).

Clinical management of ocular AdV infections is currently palliative, targeted to provide relief of symptoms. There is currently no approved treatment for adenoviral ocular infection. Antiviral drugs, such as ganciclovir and cidofovir have been tested against ocular AdV infections, without much success. Cidofovir studies have not exhibited a statistically significant improvement in the symptoms or course of the disease and was shown to cause substantial ocular toxicity, even at low doses, making it of little clinical value (Jhanji et al., supra (2015); Martinez-Aguado et al., “Antiadenovirus drug discovery: potential targets and evaluation methodologies,” Drug Discov. Today, 20(10): 1235-1242 (2015); Clement et al., “Clinical and antiviral efficacy of an ophthalmic formulation of dexamethasone povidone-iodine in a rabbit model of adenoviral keratoconjunctivitis,” Invest. Ophthalmol. Vis. Sci., 52(1): 339-344 (2011); Hillenkamp et al., “The effects of cidofovir 1% with and without cyclosporin A 1% as a topical treatment of acute adenoviral keratoconjunctivitis: a controlled clinical pilot study,” Ophthalmology, 109(5): 845-850 (2002)). Topical ganciclovir decreases AdV load experimentally but lacks efficacy in the treatment of AdV conjunctivitis in clinical trials (Yabiku et al., “Ganciclovir 0.15% ophthalmic gel in the treatment of adenovirus keratoconjunctivitis,” Arq. Bras. OftalmolI., 74: 417e21 (2011)).

With no approved antiviral drug for the treatment of ocular AdV infection, the significant global economic burden (Stenson et al., “Laboratory studies in acute conjunctivitis,” Arch. Ophthalmol., 100(8): 1275-1277 (1982)) and the presence of sight-threatening complications following ocular AdV infection, there is an urgent and unmet medical need for a safe and effective anti-AdV drug against ocular infections.

### SUMMARY OF THE INVENTION

The present invention is directed to a novel method for treating or preventing ocular viral infections in a mammal comprising topically administering to the eye an effective amount of a composition comprising filociclovir or a pharmaceutically acceptable salt thereof. The compositions and methods of the present invention are effective for treating or preventing viral infections of the eye, e.g., conjunctivitis caused by multiple strains of adenovirus (AdV) associated with diseases of the eye. In a preferred embodiment, the mammal is a human.

In addition, the compositions and methods of the present invention are also effective for treating or preventing viral ocular infections caused by a number of other viral strains including, but not limited to, viral ocular infections caused by cytomegalovirus (CMV), Epstein-Barr virus (EBV), varicella zoster virus (VZV), HHV-6A virus, HHV-6B virus, HHV-8 virus, JC virus, BK virus and a combination thereof by the topical administration to the eye of a composition comprising a pharmaceutically acceptable carrier and filociclovir or a pharmaceutically acceptable salt thereof.

In a preferred embodiment, the present invention is directed to the use of filociclovir in a method for treating or preventing an ocular viral infection caused by adenoviral strains comprising topically administering to the eye a composition comprising filociclovir or a pharmaceutically acceptable salt thereof formulated in an ophthalmically acceptable composition. The method of the present invention is suitable for the treatment of an adenoviral infection of the eye by any number of adenoviral strains known to cause infections of the eye including, adenovirus 1, 2, 3, 4, 5, 6, 7, 7a, 8, 9, 10, 11, 13, 14, 15, 16, 17, 19/64 (hereinafter adenovirus 64), 20, 21, 22, 23, 24, 25, 26, 27, 28, 29, 30, 32, 33, 36, 37, 38, 39, 42, 43, 44, 45, 46, 47, 48, 49, 53, 54, 56, and 64. In a particularly preferred embodiment, the present invention is effective to treat adenoviral infections of the eye caused by adenovirus strains 3, 4, 5, 6, 7, 7a, 8, 37, 54, and/or 64.

In another embodiment, the present invention is directed to the use of filociclovir in a method for treating or preventing an ocular viral infection caused by multiple viral strains associated with diseases of the eye including, but not limited to, viral ocular infections caused by cytomegalovirus (CMV), Epstein-Barr virus (EBV), varicella zoster virus (VZV), adenovirus (AdV), HHV-6A virus, HHV-6B virus, HHV-8 virus, JC virus, BK virus and a combination thereof.

In another embodiment, the present invention is directed to the use of filociclovir in the manufacture of a medicament for topical administration for treating or preventing ocular viral infections in a mammal caused by adenovirus.

In another embodiment, the present invention is directed to the use of filociclovir in the manufacture of a medicament for topical administration for treating or preventing ocular viral infections in a mammal caused by cytomegalovirus (CMV), Epstein-Barr virus (EBV), varicella zoster virus (VZV), adenovirus (AdV), HHV-6A virus, HHV-6B virus, HHV-8 virus, JC virus, and BK virus.

In another embodiment, the present invention is directed to the use of filociclovir to treat or prevent ocular infections cause by adenovirus comprising topically administering to the eye an effective amount of filociclovir.

In another embodiment, the present invention is directed to the use of filociclovir to treat or prevent ocular infections cause by cytomegalovirus (CMV), Epstein-Barr virus (EBV), varicella zoster virus (VZV), adenovirus (AdV), HHV-6A virus, HHV-6B virus, HHV-8 virus, JC virus, and BK virus comprising topically administering to the eye an effective amount of filociclovir.

Also disclosed are ophthalmic pharmaceutical compositions suitable for topical application for the treatment or prevention of eye infections, the compositions comprising a therapeutically effective amount of filociclovir, or a pharmaceutically acceptable salt thereof, and a pharmaceutically acceptable carrier suitable for ophthalmic administration. The pharmaceutical compositions are suitable for use in the disclosed methods for treating or preventing ocular adenoviral and other ocular viral infections in a mammal, particularly humans.

Multiple modes of topical administration for treating or preventing ocular viral infections are contemplated for the method described herein. The pharmaceutical compositions are formulated for ocular administration, such as, for example an ophthalmic ointment, an ophthalmic gel, an ophthalmic solution, an ophthalmic suspension, to a subject or patient in need thereof.

In another embodiment, the composition comprising filociclovir may be administered to a subject in need thereof optionally in combination with one or more known antiviral, anti-inflammatory, immunomodulatory, and/or antibacterial agents. These additional agent or agents may be administered before, simultaneously with, or after administration of the filociclovir.

In preferred embodiments, the filociclovir compositions of the present invention exhibit an inhibitory concentration of ≤10 μM against infections of the eye caused by adenovirus and a cytotoxicity (CC₅₀) of ≥100 μM.

In preferred embodiments, the filociclovir compositions of the present invention exhibit an inhibitory concentration of ≤10 μM against infections of the eye by CMV, HHV-6A, HHV-6B, EBV, HHV-8, VZV and a cytotoxicity (CC₅₀) of ≥100 μM, and an inhibitory concentration of ≤50 μM against infections of the eye caused by BK virus and JC virus and a cytotoxicity (CC₅₀) of ≥100 μM.

### Definitions

As used herein, the term “ocular” and “ophthalmic” refer to standard terms in the art as a reference to the eye or eye-related matters.

As used herein, the term “ophthalmically acceptable” refers to a formulation, composition or ingredient herein that has no persistent harmful effect on the treated eye or the functioning thereof, or on the general health of the subject being treated. It will be recognized that transient effects such as minor irritation or a “stinging” sensation are common with topical ophthalmic administration of drugs and the existence of such transient effects is not inconsistent with the formulation, composition or ingredient in question being “ophthalmically acceptable” as herein defined.

As used herein, the term “topical administration” refers to application of a substance such as a drug, compound, composition, etc., to the skin or mucous membranes of a mammal in any form suitable for such a procedure, for example, in the form of a gel, liquid, or paste. In the present context, topical administration includes application of a drug, compound, composition, etc., to the surface of the eye in any manner suitable to treat or prevent a viral infection of the eye.

As used herein, the term “treat” and variations thereof, e.g., “treating”, “treatment”, refer to the administration of an agent or formulation to a clinically symptomatic individual afflicted with an adverse condition disorder, or disease, so as to effect a reduction in severity and/or frequency of symptoms, eliminate the symptoms and/or their underlying cause, and/or facilitate improvement or remediation of damage.

As used herein, the term “preventing” with respect to a condition or disorder refers to delaying or preventing the onset of such ophthalmic disorder or condition described herein, e.g., in a subject at risk of having the condition. In some embodiments, “preventing” a condition can also encompass inhibiting, decreasing, or slowing the progression or severity of the condition, e.g., in a subject being diagnosed with the condition. The onset, the progression, or severity of such disorder or condition can be determined by detecting an increase in at least one symptom associated with the condition, or a decrease in the function of the organ or organs affected by the condition.

The phrase “effective amount” or “therapeutically effective amount” as used herein refers to an amount of a compound described herein, or a composition comprising the compound, which is effective for producing some desired therapeutic effect in at least a sub-population of cells in a subject at a reasonable benefit/risk ratio applicable to any medical treatment. For example, a therapeutically effective amount of a compound or a composition comprising the compound can be an amount sufficient to produce a statistically significant, measurable change in at least one symptom of conjunctivitis as described herein.

As used herein, the term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids”, includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

A composition or method described herein as “comprising” (or “comprises”) one or more named elements or steps is open-ended, meaning that the named elements or steps are essential, but other elements or steps may be added within the scope of the composition or method. To avoid prolixity, it is also understood that any composition or method described as “comprising” one or more named elements or steps also describes the corresponding, more limited, composition or method “consisting essentially of” (or “consists essentially of”) the same named elements or steps, meaning that the composition or method includes the named essential elements and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other element or step. In any composition or method disclosed herein, known or disclosed equivalents of any named essential element or step may be substituted for that element or step, respectively.

Filociclovir (also referred to as cyclopropavir, 2-amino-9-{(Z)-[2,2-bis(hydroxymethyl)cyclopropylidene]methyl}-3,9-dihydro-6H-purin-6-one), and salts thereof, is a methylenecyclopropane nucleoside analogue represented by the following structure:

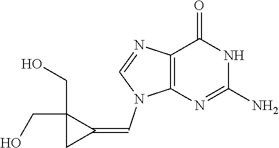 [SMILES: Nc1nc2c(ncn2/C=C2/CC2(CO)CO)c(=O)[nH]1]

As used herein, the term “subject” can be a human, non-human primate, horse, pig, rabbit, dog, sheep, goat, cow, cat, guinea pig or rodent. A “patient” or “subject in need thereof” refers to a mammal afflicted with a disease or disorder. The term “patient” includes human and veterinary subjects.

### BRIEF DESCRIPTION OF THE DRAWINGS

FIG. 1 is a dose response curve showing antiviral activity (closed symbols) and cytotoxicity (open symbols) of filociclovir against adenovirus 5 (AdV5).

FIG. 2 is a dose response curve showing the antiviral activity and cytotoxicity of filociclovir against adenovirus 6 (AdV6).

FIG. 3 is an immunofluorescence assay of A549 cells infected with adenovirus 5 and treated with filociclovir.

FIG. 4 is an immunofluorescence assay of A549 cells infected with adenovirus 6 and treated with filociclovir.

FIG. 5 shows the bioavailability of filociclovir administered orally (PO) or intravenously (IV) to hamsters.

FIGS. 6A and 6B show the percent survival (6A) and mean body weight changes (6B) of Syrian hamsters infected with AdV6 and treated with 10 mg/kg, 30 mg/kg, 60 mg/kg, or 100 mg/kg filociclovir. 6A: Survival. AdV6+Vehicle v. AdV6+filociclovir 10 mg/kg or 30 mg/kg p=0.0124 (log rank). 6B: Mean body weight changes. Group means and standard error of the mean are shown. After moribund animals were sacrificed from a group, there were no means calculated for that group. AdV6+Vehicle vs. AdV6+filociclovir 10 mg/kg or 30 mg/kg p<0.0001 (two-way ANOVA); AdV6+filociclovir at 10 mg/kg vs. Vehicle+Vehicle or AdV6+filociclovir 30 mg/kg p=0.0286.

FIG. 7 shows the transaminase (ALT) levels of the Syrian hamsters at 5 days post challenge. Treatment with filociclovir mitigates liver pathology. The symbols indicate values from individual animals; the horizontal bar represents the geometric mean. The empty symbols in the AdV6+Vehicle group denote samples that were collected from moribund animals.

FIG. 8 shows the level of AdV burden in the livers of the Syrian hamsters 5 days post challenge. Treatment with filociclovir inhibits AdV6 replication in the liver. The symbols indicate values from individual animals; the horizontal bar represents the geometric mean. NQ: not quantifiable; ND: not detectable.

FIG. 9 shows the comparison in adenovirus positive cultures from rabbits treated with 1% filociclovir (1A), 0.5% filociclovir (1B), 0.5% cidofovir (1C), and vehicle (control) (1D) over a 14-day period.

FIG. 10 shows the duration of adenovirus shedding from rabbits treated with 1% filociclovir (1A), 0.5% filociclovir (1B), 0.5% cidofovir (1C), and vehicle (control) (1D) over a 14-day period.

FIG. 11 shows median adenovirus ocular titers from rabbits treated with 1% filociclovir (1A), 0.5% filociclovir (1B), 0.5% cidofovir (1C), and vehicle (control) (1D) over a 14-day period.

### DETAILED DESCRIPTION OF THE INVENTION

Human adenoviruses belong to the adenoviridae family, which is distinct from the herpesviridae family. For example, adenoviruses are double-stranded DNA viruses but, unlike herpesviruses, they are non-enveloped with an icosahedral nucleocapsid, and carry elongated fiber proteins on their outer surface. Adenoviruses are classified into seven species designated human adenovirus A to G. These viruses mainly infect the conjunctiva, i.e., the mucous membrane that covers the front of the eye and lines the inside of the eyelids, upper or lower respiratory tracts, or gastrointestinal tract, causing a whole array of clinical manifestations that are distinct from those caused by herpesviruses. Adenoviruses are not known to encode virus-encoded kinases, which implies a novel mechanism for filociclovir activation.

Acute conjunctivitis is a common condition, estimated to affect 6 million people each year in the United States (Udeh et al., 2008, supra) with adenoviruses being the most common cause. Adenoviral conjunctivitis can cause significant discomfort and lost productivity. Although mostly self-limiting, in some cases it can lead to complications from long-term immune-mediated sequelae (Ford et al., 1987, supra). No antiviral drug has been approved for treating adenovirus-associated conjunctivitis.

In addition to the Adenoviridae family, other viral strains known to be responsible for conjunctivitis include, but are not limited to, cytomegalovirus (CMV), Epstein-Barr virus (EBV), varicella zoster virus (VZV), HHV-6A virus, HHV-6B virus, HHV-8 virus, JC virus, and BK virus. An individual may also contract conjunctivitis from exposure to a combination of these viruses.

Therefore, currently there is a need for a safe and effective treatment for conjunctivitis, and in particular conjunctivitis known to be caused by multiple strains of adenovirus.

The data described herein, utilizing both an in vitro assay and an in vivo art-recognized rabbit model of conjunctivitis, demonstrate that filociclovir provides a safe and effective therapeutic for treating and preventing viral associated diseases of the eye when administered topically to the affected area, namely when applied to the eye. In a preferred embodiment, the viral infection is caused by an adenovirus strain.

As described herein, it has been demonstrated that filociclovir exhibits a dose-dependent inhibition of adenovirus infection with IC₅₀ values ≤5 μM and a minimal mammalian cytotoxicity (CC₅₀) of preferably ≥100 μM.

Therefore, in one aspect, the present invention is directed to the discovery of a novel method for treating and/or preventing ocular adenovirus infections in a mammal comprising the topical administration of the small molecule inhibitor filociclovir (FCV) formulated in a pharmaceutically acceptable carrier or excipient suitable for ophthalmic administration. The data described herein demonstrate that filociclovir is a safe and effective inhibitor of a wide variety of adenoviral strains associated with conjunctivitis including adenovirus 3, 4, 5, 6, 7, 7a, 8, 37, 54, and 64.

Advantageously, the Adenoviridae family of viruses are very closely related and the demonstrated antiviral activity of filociclovir against a number of these strains is strong evidence that filociclovir will have the same inhibitory effect against all viruses within the Adenoviridae family associated with diseases of the eye. Therefore, in another embodiment, the present invention is directed to the use of filociclovir in a method for treating or preventing ocular infection of a mammal by adenovirus strains 1, 2, 3, 4, 5, 6, 7, 7a, 8, 9, 10, 11, 13, 14, 15, 16, 17, 20, 2122, 23, 24, 25, 26, 27, 28, 29, 30, 32, 33, 36, 37, 38, 39, 42, 43, 44, 45, 46, 47, 48, 49, 53, 54, 56, and/or 64. In a particularly preferred embodiment, the present invention is effective to treat adenoviral infections of the eye caused by adenovirus strains 3, 4, 5, 6, 7, 7a, 8, 37, 54, and/or 64. The methods described herein comprise the topical administration of a composition comprising filociclovir or a pharmaceutically acceptable salt thereof formulated in an ophthalmic pharmaceutically acceptable carrier or excipient.

Therefore, in one embodiment, the present method is directed to the treatment and prevention of conjunctivitis caused by adenovirus 3, comprising the topical administration of filociclovir, or a pharmaceutically acceptable salt thereof, formulated in a pharmaceutically acceptable carrier or excipient formulated in an ophthalmologically acceptable composition or carrier.

In another embodiment, the present method is directed to the treatment and prevention of conjunctivitis caused by adenovirus 4, comprising the topical administration of filociclovir, or a pharmaceutically acceptable salt thereof, formulated in a pharmaceutically acceptable carrier or excipient formulated in an ophthalmologically acceptable composition or carrier.

In another embodiment, the present method is directed to the treatment and prevention of conjunctivitis caused by adenovirus 5, comprising the topical administration of filociclovir, or a pharmaceutically acceptable salt thereof, formulated in a pharmaceutically acceptable carrier or excipient formulated in an ophthalmologically acceptable composition or carrier.

In another embodiment, the present method is directed to the treatment and prevention of conjunctivitis caused by adenovirus 6, comprising the topical administration of filociclovir, or a pharmaceutically acceptable salt thereof, formulated in a pharmaceutically acceptable carrier or excipient formulated in an ophthalmologically acceptable composition or carrier.

In another embodiment, the present method is directed to the treatment and prevention of conjunctivitis caused by adenovirus 7, comprising the topical administration of filociclovir, or a pharmaceutically acceptable salt thereof, formulated in a pharmaceutically acceptable carrier or excipient formulated in an ophthalmologically acceptable composition or carrier.

In another embodiment, the present method is directed to the treatment and prevention of conjunctivitis caused by adenovirus 7a, comprising the topical administration of filociclovir, or a pharmaceutically acceptable salt thereof, formulated in a pharmaceutically acceptable carrier or excipient formulated in an ophthalmologically acceptable composition or carrier.

In another embodiment, the present method is directed to the treatment and prevention of conjunctivitis caused by adenovirus 8, comprising the topical administration of filociclovir, or a pharmaceutically acceptable salt thereof, formulated in a pharmaceutically acceptable carrier or excipient formulated in an ophthalmologically acceptable composition or carrier.

In another embodiment, the present method is directed to the treatment and prevention of conjunctivitis caused by adenovirus 37, comprising the topical administration of filociclovir, or a pharmaceutically acceptable salt thereof, formulated in a pharmaceutically acceptable carrier or excipient formulated in an ophthalmologically acceptable composition or carrier.

In another embodiment, the present method is directed to the treatment and prevention of conjunctivitis caused by adenovirus 54, comprising the topical administration of filociclovir, or a pharmaceutically acceptable salt thereof, formulated in a pharmaceutically acceptable carrier or excipient formulated in an ophthalmologically acceptable composition or carrier.

In another embodiment, the present method is directed to the treatment and prevention of conjunctivitis caused by adenovirus 64, comprising the topical administration of filociclovir, or a pharmaceutically acceptable salt thereof, formulated in a pharmaceutically acceptable carrier or excipient formulated in an ophthalmologically acceptable composition or carrier.

In another embodiment, the present invention is directed to the discovery of a novel method for treating and/or preventing ocular infections caused by cytomegalovirus (CMV), Epstein-Barr virus (EBV), varicella zoster virus (VZV), HHV-6A virus, HHV-6B virus, HHV-8 virus, JC virus, and BK virus in a mammal comprising the topical administration of filociclovir formulated in a pharmaceutically acceptable carrier or excipient suitable for ophthalmic administration. As set forth in the table below, filociclovir has been shown to be an effective inhibitor against a number of adenovirus and other viral strains associated with diseases of the eye with EC₅₀ values of ≤10 μM against infection by CMV, HHV-6A, HHV-6B, EBV, HHV-8, VZV and EC₅₀ values of ≤50 μM against infection by BK virus and JC virus.

| | Type | | | | | | | |
|---|---|---|---|---|---|---|---|---|
| | β | | | γ | | α | Polyomavirus | |
| | Herpesvirus | | | Herpesvirus | | Herpesvirus | BK | JC |
| Strain | CMV‡ | HHV-6A* | HHV-6B† | EBV† | HHV-8† | VZV‡ | Virus‡ | Virus‡ |
| Filociclovir | 1.0 ± 0.6 | 1.3 | 2.3 ± 1 | 1.0 ± 0.94 | 5.6 ± 5 | 1.5 ± 1.6 | 34 ± 17 | 46 ± 44 |
| EC₅₀ (μM) | | | | | | | | |

| | Type | | | | |
|---|---|---|---|---|---|
| | Adenovirus | | | | |
| Strain | AdV 4 | AdV 5‡ | AdV 6 | AdV 7 | AdV 8 |
| Filociclovir | 2.4 | 2.2 ± 0.4 | 2 | 1 | 2 |
| EC₅₀ (μM) | | | | | |

*Pritchard et al. Antimicrobial Agents and Chemotherapy, 2013, 57: 3518-3527
‡Hartline et al. Antiviral Research, 2018, 159: 104-112
‡Keith et al. Antiviral Research, 2018, 159: 122-129

In another embodiment, the present invention is directed to the use of filociclovir in the manufacture of a medicament for use in a method for treating or preventing ocular infections caused by adenovirus, the method comprising topical administration to the eye of a composition comprising filociclovir, or a pharmaceutically acceptable salt thereof formulated in an ophthalmic pharmaceutically acceptable carrier or excipient. In a preferred embodiment, the method is effective to treat ocular infections caused by adenoviral strains 1, 2, 3, 4, 5, 6, 7, 7a, 8, 9, 10, 11, 13, 14, 15, 16, 17, 20, 21, 22, 23, 24, 25, 26, 27, 28, 29, 30, 32, 33, 36, 37, 38, 39, 42, 43, 44, 45, 46, 47, 48, 49, 53, 54, 56, and/or 64. In a particularly preferred embodiment, the method is effective to treat adenoviral infections of the eye caused by adenovirus strains 3, 4, 5, 6, 7, 7a, 8, 37, 54, and/or 64.

In another embodiment, the present invention is directed to the use of filociclovir in the manufacture of a medicament for use in a method for treating or preventing ocular infection caused by cytomegalovirus (CMV), Epstein-Barr virus (EBV), varicella zoster virus (VZV), HHV-6A virus, HHV-6B virus, HHV-8 virus, JC virus, and BK virus in a mammal comprising the topical administration of filociclovir or a pharmaceutically acceptable salt thereof formulated in a pharmaceutically acceptable carrier or excipient suitable for ophthalmic administration.

In another embodiment, the present invention is directed to the use of filociclovir to treat adenovirus-related ocular infections in a mammal comprising the topical administration of a composition comprising filociclovir or a pharmaceutically acceptable salt thereof formulated in a pharmaceutically acceptable carrier or excipient suitable for ophthalmic administration.

Multiple modes of administration suitable for application to the eye are contemplated for delivery of the ophthalmic composition of the present invention to an infected individual. The active ingredients can be administered in the conjunctival sack as eye drops, ointments, creams, gels, sustained release carriers, slow dissolving capsules placed in the conjunctival sack, via administration or release from a contact lens, subconjunctivally by injection, or intravitreally by injection, by preparing a suitable formulation of the active ingredient and utilizing procedures well known to those skilled in the art. In one embodiment, the formulations are prepared with suitable nontoxic pharmaceutically acceptable ingredients. These ingredients are known to those skilled in the preparation of eye drops, eye ointments, subconjunctival and intravitreal injections. Some of these ingredients can be found in Remington's Pharmaceutical Sciences, 17th edition, 1985, a standard reference in the field. The choice of suitable carriers may be dependent upon the exact nature of the eye drops, eye ointments, subconjunctival, intravitreal dosage form desired, e.g. solutions, sprays, drops, gels, pastes, patches.

By “suitable for application to the eye” is meant that any component of the formulation does not cause a long-lasting deleterious effect on the eye or the subject being treated. Transient effects such as minor irritation or ‘stinging’ upon administration may occur without long-lasting deleterious effects. Modes of administration of the filociclovir composition of the present invention to eye of a subject suffering from viral conjunctivitis include the following:

### A. Topical Ophthalmic Drug Forms:

Liquid Ophthalmic Drug Forms
Eye Drops
Ophthalmic solutions
Suspensions/nanosuspensions
Emulsions/Microemulsions

Semisolid Ophthalmic Drug Forms
In Situ Gels
Eye ointments

Solid Ophthalmic Drug Forms
Contact lenses coated with drugs
Ocular inserts
Soluble ophthalmic drug inserts (soluble eye inserts in the form of small oval wafers, produced from acrylamide, N-vinylpyrrolidone, and ethyl acrylate.)
Minidiscs/Ocular therapeutic system (a profiled, convex outside, concave from the side of contact with eye surface, dosage form similar to a contact lens with 4-5 mm diameter.)
Artificial tear inserts
Collagen shield
Minitablets (biodegradable, solid drug forms, that, after application to conjunctival sac, transit into gels, which extends the time period of contact between active ingredient and the eyeball surface)

Multicompartment Drug Delivery Systems
Nanoparticles and Microparticles
Micelles/nanomicelles
Liposomes
Niosomes (chemically stable, biodegradable, biocompatible, nonimmunogenic, built of nonionic surfactants, two-layered carriers used for both hydrophilic and hydrophobic particles) and Discosomes (modified forms of niosomes)
Dendrimers

Other Ophthalmic Drug Forms
Filter paper strips
Sprays
Ocular iontophoresis
   B. Injection (intravitreal, subconjunctival, retrobulbar, peribulbar, sub-tenon, intracameral)

### C. Implants

### D. Microneedles

The concentration of filociclovir in the pharmaceutical compositions described herein is in the range of about 0.001% to 30%, or about 0.001% to 20% or about 0.001 to 10% weight of the total composition. In another embodiment, the amount of filociclovir is in the range of about 0.001-5% weight of the total pharmaceutical composition.

Pharmaceutically acceptable salts for the filociclovir formulations described herein include the gluconate, lactate, acetate, tartarate, citrate, phosphate, maleate, borate, nitrate, sulfate, and hydrochloride salts. The salts of the compounds described herein can be prepared, for example, by reacting the base compound with the desired acid in solution. After the reaction is complete, the salts are crystallized from solution by the addition of an appropriate amount of solvent in which the salt is insoluble. In some embodiments, the hydrochloride salt is made by passing hydrogen chloride gas into an ethanolic solution of the free base.

An aqueous suspension or solution/suspension of the invention for ophthalmic administration can contain one or more polymers as suspending agents. Exemplary polymers include, but are not limited to, water-soluble polymers such as cellulosic polymers, e.g., hydroxypropyl methylcellulose, and water-insoluble polymers such as cross-linked carboxyl-containing polymers. In some embodiments, the polymer may include hydroxypropyl methylcellulose, guar gum, carboxyvinyl polymers (acrylic acid polymer), hydroxyethyl cellulose, carboxymethylcellulose, poly(methylmethacrylate), polyacrylamide, polycarbophil, polyethylene oxide, acrylic acid/butyl acrylate copolymer, sodium alginate and dextran.

Any excipients known to one skilled in the art may be included in the ophthalmic formulations of the present invention to increase retention of the composition in an eye. Preferably, the composition comprising filociclovir, upon topical application to the eye, will be retained for a time on the applied surface allowing diffusion of the compound into the ocular environment allowing the anti-viral activity of the filociclovir to be effective and not quickly washed away by the ocular fluids. Exemplary excipients include, but are not limited to, monomeric polyols, such as, glycerol, propylene glycol, ethylene glycol; polymeric polyols, such as, polyethylene glycol, hydroxypropylmethyl cellulose (“HPMC”), carboxy methylcellulose sodium, hydroxy propylcellulose (“HPC”), dextrans, such as, dextran 70; water soluble proteins, such as gelatin; and vinyl polymers, such as, polyvinyl alcohol, polyvinyl/pyrrolidone povidone and carbomers, such as, carbomer 934P, carbomer 941, carbomer 940, carbomer 974; phydroxyethylcellulose; methylcellulose; polyvinylpyrrolidone; polysaccharides, such as hyaluronic acid and its salts; chondroitin sulfate and its salts; dextrans; various polymers of the cellulose family; vinyl polymers; and acrylic acid polymers.

The ophthalmic compositions may include gelling agents or viscosity control agents include gelling agents that increase viscosity when they come into contact with lacrimal fluid, for example, lacrimation caused by blinking or tears. Such gelling agents may be used to reduce loss of the composition by lacrimal drainage and allow the composition to have increased residence time and therefore adsorption in the eye or epithelial layer of the eyelids. Suitable gelling agents include gellan gum, especially low acetylated gellan gum, alginate gum, or chitosan. Viscosity control agents include natural polysaccharides, natural gums, modified natural polymers, synthetic polymers, proteins and synthetic polypeptides that are capable of increasing viscosity and are ophthalmically acceptable. In some embodiments, at least one viscosity control agent is a mucomimetic. In another embodiment, at least one viscosity control agent is a carboxyvinyl polymer.

The filociclovir composition may be formulated in a simple aqueous solution or carrier or formulated to have physiologically compatible osmolality and pH, for example, by including ophthalmically acceptable salts and buffering agents, and other components such as preservatives, gelling agents, viscosity control agents, ophthalmic lubricating agents, mucoadhesive polymers, surfactants, antioxidants and the like in a solution, gel, lotion, or ointment suitable for topical administration to the eye.

An ophthalmically acceptable composition or carrier comprises an aqueous solution, a non-aqueous solution, or an emulsion, etc. (for example, water, oil, wax, grease or petrolatum or a combination thereof). Exemplary aqueous carriers include, but are not limited to water, buffered water, 0.8% saline, 0.3% glycine, hyaluronic acid, phospholipid carriers or artificial tears carriers, or mixtures of such carriers and the like. As used herein, the term “phospholipid” refers to the phospholipids of the phospholipid carrier. Exemplary phospholipid carriers and artificial tears carriers include but are not limited to those described in U.S. Pat. No. 6,645,978, which is incorporated herein.

In some embodiments, the ophthalmic carrier may be a salve or ointment carrier. Such salves or ointments typically comprise one or more 4-aminoquinoline compounds dissolved or suspended in a sterile pharmaceutically acceptable salve or ointment base, such as a mineral oil-white petrolatum base. In salve or ointment compositions, anhydrous lanolin may also be included in the formulation. Thimerosal or chlorobutanol may also be added to such ointment compositions as antimicrobial agents.

In yet another embodiment of the invention, the ophthalmic carrier may be olive oil, arachis oil, castor oil, polyoxyethylated castor oil, mineral oil, petroleum jelly, dimethyl sulphoxide, an alcohol, liposome, silicone fluid and mixtures thereof as disclosed in U.S. Pat. No. 6,254,860. In one embodiment, the ophthalmic carrier is a conjunctival insert such as described in U.S. Pat. No. 6,217,896.

In some embodiments, the compositions described herein may comprise an ophthalmically acceptable xanthine derivative such as caffeine, theobromine, or theophylline as disclosed in U.S. Pat. No. 4,559,343, incorporated herein by reference.

The compositions described herein may optionally comprise tonicity imparting agents such as sodium chloride and concentrated glycerol and viscosity imparting agents such as sodium carboxymethylcellulose, microcrystalline cellulose, polyvinylpyrrolidone, polyvinyl alcohol.

In some embodiments, the compositions described herein may comprise suitable absorption enhancers, such as surfactants, bile acids; stabilizing agents for example antioxidants such as bisulfites and ascorbates; and/or metal chelating agents, such as sodium edetate; and drug solubility enhancers, such as polyethylene glycols.

In another embodiment of the invention, the ophthalmic compositions described herein may comprise a surfactant such as polyoxyethylene fatty acid glycerides, vegetable oils, e.g., polyoxyethylene (60) hydrogenated castor oil; and polyoxyethylene alkylethers and allylphenyl ethers, e.g., octoxynol 10, octoxynol 40, a polyoxyethylene fatty acid ester, polyoxyethylene alkylphenyl ether, and polyoxyethylene alkyl ether, or mixtures thereof or a thickening agent such as a carboxyvinyl polymer, polyvinyl polymer, and polyvinylpyrrolidones, as described in U.S. Pat. No. 5,951,971.

The compositions described herein may comprise at least one ophthalmically acceptable acid having at least two dissociable hydrogen groups that may be included in a polymer-containing composition as interactive agents to retard the release of the drug through inhibition of erosion of the polymer, as disclosed in International Patent Publication No. WO 95/03784, incorporated herein by reference. Other exemplary interactive agents include, but are not limited to, boric, lactic, orthophosphoric, citric, oxalic, succinic, tartaric and formic glycerophosphoric acids.

Ophthalmic solutions can be prepared using distilled water, an aqueous base, or any other acceptable base; tonicity agents; buffers such as sodium phosphate and sodium acetate; surfactants such as polyoxyethylene sorbitan monooleate, stearic polyoxyl 40, and polyoxyethylene hydrogenated castor oil; stabilizers such as sodium citrate and sodium edetate; preservatives such as benzalkonium chloride, thimerosal, chlorobutanol, sodium chloride, boric acid, parahydroxybenzoic acid esters (sorbate, benzoate, propi-onate), chlorobutanol, benzyl alcohol, mercurials, paraben such as propyl 4-hydroxybenzoate (or propylparaben), methyl-P-Hydroxybenzoate (or methylparaben), and mixtures thereof. In some embodiments, preservatives include benzalkonium chloride or thimerosal.

Suitable ophthalmically acceptable salts useful as osmolality agents include salts having sodium, potassium, or ammonium cations and chloride, citrate, ascorbate, borate, phosphate, bicarbonate, sulfate, thiosulfate or bisulfite ions. Examples of suitable salts include sodium chloride, potassium chloride, sodium thiosulfate, sodium bisulfate and ammonium sulfate. Excipients suitable to adjust osmolality include sugars, for example dextrose, lactose, xylitol, mannitol and glycerine.

Suitable ophthalmically acceptable pH adjusting agents and/or buffering agents include acids such as acetic, boric, citric, lactic, phosphoric and hydrochloric acids, bases such as sodium hydroxide, sodium phosphate, sodium borate, sodium citrate, sodium acetate, sodium lactate and trishydroxymethylaminomethane, and buffers such as citrate-dextrose, sodium bicarbonate and ammonium chloride, or an amino acid. Such an acid, base and/or buffer may be included in an amount required to maintain pH of the composition in an ophthalmically acceptable range.

Suitable preservatives include stabilized ammonium compounds such as benzalkonium chloride, cetyltrimethylammonium chloride and cetylpyridinium chloride, murcuric compounds such as phenyl mercuric acetate, imidazolidinyl urea, parabens such as methyl paraben ethyl paraben, propyl paraben or butyl paraben; phenoxyethanol, chlorophenoxyethanol, phenoxypropanol, chlorobutanol, chlorocresol, phenylethyl alcohol, ethylenediamine tetraacetic acid, sorbic acid and salts thereof.

Suitable lubricating agents to promote lacrimation include polyvinyl alcohol, methyl cellulose, hydroxypropyl methylcellulose and polyvinylpyrrolidone. Suitable mucoadhesive polymers include hydroxypropyl methylcellulose, carboxymethylcellulose, poly(methylmethacrylate), polyacrylamide, polycarbophil, polyethyl oxide, sodium alginate and dextrin. Suitable ophthalmically acceptable surfactants include non-ionic surfactants such as polyoxyethylene fatty acid glycerides and vegetable oils including polyoxyethylene (60) hydrogenated castor oil, polyoxyethyl alkylethers and alkylphenyl ethers such as octoxynol 10 and octoxynol 40.

In some embodiments, the ophthalmic composition further comprises a penetration enhancer, preferably present in an amount in the range of about 0.001 wt. % to about 5 wt. %.

In some embodiments, the compositions described herein may include at least one antioxidant to enhance chemical stability. Exemplary antioxidants include, but are not limited to, ascorbic acid and derivatives, sodium metabisulfite, vitamin E and analogs thereof and butylated hydroxyanisole (BHA).

In some embodiments, the compositions described herein further comprise a vasoconstrictor. Exemplary vasoconstrictors include, but are not limited to, tetrahydrozoline, ephedrine, naphazoline, phenylephrine, and/or mixtures thereof.

In some embodiments, the ophthalmic composition described herein comprises an ophthalmic wetting agents and/or ophthalmic diluting agents. Wetting agents commonly used in ophthalmic solutions include carboxymethylcellulose, hydroxypropyl methylcellulose, glycerin, mannitol, polyvinyl alcohol or hydroxyethylcellulose. Diluting agents include water, distilled water, saline solution, sterile water, artificial tears, etc. wherein the wetting agent is present in an amount of about 0.001% to about 30%.

In another embodiment, the ophthalmic pharmaceutical composition further comprises one or more additional active ophthalmic pharmaceutical agents such as anti-inflammatory agents, antibiotics, antiviral, ocular hypotensive agents, local anesthetic agents, cycloplegics, or pupillary dilators, which are used in the treatment of diseases of the eye.

The compositions of the present invention described herein comprising filociclovir may also be administered via a contact lens by adding an ophthalmically suitable formulation comprising filociclovir directly to a contact lens or to contact lens solutions including washing, rinsing, storing, disinfecting, rewetting, and lubricating solutions for contact lenses. This allows for the filociclovir composition to diffuse out of the contact lens and directly onto the eye for release into the ocular environment and allows for the composition to be delivered over a longer period of time than without the contact lens. Therefore, in another aspect, the present invention provides a contact lens solution comprising filociclovir or a pharmaceutically acceptable salt thereof. The pH of the solution should be adjusted to be compatible with the eye and the contact lens, for example, the pH of the solution should be between 6.0 and 8.0, preferably between 6.8 and 7.9, and most preferably between 7.0 and 7.6.

The compositions described herein may also be administered via a biocompatible and implantable controlled-release drug delivery device as taught in U.S. Pat. No. 6,331,313. The compositions described herein may also be administered in sustained release forms or from sustained release drug delivery systems which are described in Remington's Pharmaceutical Sciences, 17th edition, 1985, Mack Publishing Company, Easton, Pa., and International Program on Chemical Safety (IPCS).

Ophthalmic dosing of the filociclovir composition will depend on the severity of the condition. For example, the formulation may be administered to the eye 1 to 6 times per day, 1 to 4 times per day, 1 to 3 times per day, or at least once per day. Preventative dosing may occur one or more times per day over the course of 1 to 30 days, 1 to 20 days, 1 to 10 days, etc. for as long as the risk of contracting conjunctivitis persists.

In another embodiment, the present invention is directed to the use of filociclovir for the treatment or prevention of conjunctivitis in combination with one or more known antiviral agents. The additional antiviral agent or agents may be administered before, simultaneously with, or after administration of the filociclovir. Suitable antiviral agents for use in the present method include, but are not limited to, cidofovir, acyclovir, valacyclovir, famciclovir, ganciclovir, zalcitabine, alovudine, stampidine, ribavirin cyclosporine 2′,3′-dideoxycytidine (ddC), 6-azacytidine, (S)-HPMPC, (S)-HPMPA and 2-nor-cyclic GMP.

In another embodiment, the present invention is directed to the use of filociclovir for the treatment or prevention of conjunctivitis in combination with one or more known anti-inflammatory agents. The additional anti-inflammatory agent or agents may be administered before, simultaneously with, or after administration of the filociclovir. Suitable anti-inflammatory agents suitable for use in the present method include salicylates such as aspirin, diflunisil and salsalate; propionic acid derivates such a ibuprofen dexibuprofen, fenoprofen, ketaprofen, dexketoprofen flubiprofen, oxaprozin and laxoprofen, acetic acid derivatives such as indomethacin, tolmetin, sulindac, etodolac, keorolac, diclofenac, and nabumetone, enolic acid derivatives such as piroxicam, maloxicam, tenoxicam, droxicam, lomoxican and isoxicam, fenamic acid derivatives such as mefenamic acid, meclofenamic acid, flufenamic acid and tolfenamic acid, COX-2 inhibitors such as celecoxib, rofecoxib, valdecoxib, parecoxib, lumiracoxib, etoricoxib and firocoxib.

In another embodiment, the present invention is directed to the use of filociclovir with one or more known immunomodulatory agents. The additional immunomodulatory agent or agents may be administered before, simultaneously with, or after administration of the filociclovir. Immunomodulatory agents suitable for use in the present method include cyclosporin A, mycophenolate mofetil, tacrolimus, rapamycin, and leflunomide.

In another embodiment, the present invention is directed to the use of filociclovir in a method for the treatment or prevention of conjunctivitis in combination with one or more known antibacterial agents. The additional antibacterial agent or agents may be administered before, simultaneously with, or after administration of the filociclovir. Antibacterial agents suitable for use in the present method include ciprofloxacin, norfloxacin, trimethoprim-polymyxin B, levofloxacin, natamycin, tobramycin, bacitracin, trifluridine, gatifloxacin, moxifloxacin, besifloxacin, azithromycin, chloramphenicol, bacitracin-polymyxin B, povidone iodine, sulfacetamide sodium, erythromycin, gentamicin, bacitracin-neomycin-polymyxin B, gramicidin-neomycin-polymyxin B, ofloxacin, and oxytetracycline-polymyxin B.

Different concentrations of compounds described herein may achieve similar results, with the compounds described herein administered, typically and without limitation, from one to ten times daily, including 2, 3, 4, 5, 6, 7, 8, 9 and 10 times daily. The amount (e.g., number of drops of drug product) of the drug product administered to the patient (typically one or two drops per eye per dose when a dropper is used), also may vary depending on the ocular dispenser used to administer the drug product and the concentration of the binding reagent and, where appropriate, anti-inflammatory agent in the drug product. A person of average skill in the pharmaceutical and medical arts will appreciate that it will be a matter of simple design choice and optimization to identify a suitable dosage regimen for treatment of any given ocular infection or prevention of an ocular infection.

In one aspect, the invention relates to a kit comprising at least filociclovir according to the present invention, or a pharmaceutically acceptable salt, solvate, or polymorph thereof; and one or more of:
a) optionally at least one additional agent known to have one or more of the following: antiviral activity, anti-inflammatory activity, immunomodulatory activity, antibacterial activity;
b) instructions for treating an adenovirus-related disease;
c) instructions for administering the compound in connection with treating a adenovirus infection; or
d) instructions for administering the compound with at least one agent known to treat an adenovirus-related disease.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound of the present invention and/or product and another component for delivery to a patient.

In a further aspect, the kit further comprises a plurality of dosage forms, the plurality comprising one or more doses; wherein each dose comprises an amount of the compound and the agent known to have antiviral activity. In another aspect, the kit further comprises a plurality of dosage forms, the plurality comprising one or more doses; wherein each dose comprises an effective amount of the compound and the agent known to have antiviral activity.

In a further aspect, an effective amount is a therapeutically effective amount. In a still further aspect, an effective amount is a prophylactically effective amount.

The data described below, using both in vitro and in vivo models for conjunctivitis, demonstrate that filociclovir is effective to treat or prevent adenoviral infections associated with conjunctivitis.

The data described herein also indicate that filociclovir is a pan-adenoviral therapeutic, with added broad-spectrum activity against herpes viruses as well as polyoma viruses; thus, therapeutic administration of filociclovir is effective to treat or prevent infection by multiple strains of adenovirus as well as a number of other viruses. As described below, using several assays including a cytopathic effects (CPE) reduction assay, a neutral red survival assay, and an immunofluorescence assay, filociclovir is demonstrated to be effective at preventing infection by multiple strains of adenovirus, as well as other viruses.

### EXAMPLES

The following Examples have been included to illustrate modes of the presently disclosed subject matter. In light of the present disclosure and the general level of skill in the art, those of skill will appreciate that the following Examples are intended to be exemplary only and that numerous changes, modifications, and alterations can be employed without departing from the invention as presently disclosed.

### Example 1. Cytopathic Effects (CPE) Reduction Assay to Measure Inhibition of Adenovirus 5 (AdV5) by Filociclovir

### Preparation and Culture of Human Foreskin Fibroblast (HFF) Cells

Human foreskin tissue was obtained from the University of Alabama at Birmingham tissue procurement facility with approval from the Institutional Review Board. The tissue was stored at 4° C. in cell culture medium consisting of minimum essential media (MEM) with Earle's salts supplemented with 10% fetal bovine serum (FBS; HyClone, Inc., Logan, Utah) and standard concentration of L-glutamine, amphotericin B (Fungizone), and vancomycin. The tissue was then placed in a phosphate-buffered saline solution, minced, and rinsed to remove the red blood cells. Tissue fragments were then resuspended in a trypsin-EDTA solution and incubated at 37° C. to disperse the cells, which were then collected by centrifugation. Cell pellets were then resuspended in 4 ml culture medium, placed in a 25-cm² tissue culture flask, and incubated at 37° C. for 24 h. The culture medium was then replaced with fresh medium and condition of the cells was monitored daily until a confluent cell monolayer was formed. The HFF cells were then expanded through serial passages in standard growth medium of MEM with Earle's salts supplemented with 10% FBS, L-glutamine, penicillin, and gentamicin. Each lot of cells was confirmed to be free of mycoplasma infection and routinely passaged or used for assays at or before passage 10.

### Antiviral Assay

Antiviral and cytotoxicity data were obtained in a series of three to five separate experiments for each virus to provide an accurate estimate of antiviral activity and statistical data. Each assay included positive and negative control compounds as well as infected and uninfected controls to ensure the integrity of the experiment. The concurrent assessment of cytotoxicity was performed in each assay plate using the same number of cells and equivalent levels of compound exposure so that accurate selective index (SI) values could be obtained. All liquid handling steps were performed on a BioMek 4000 and significantly increased the efficiency of the assays and reduced hands-on time of analysts.

The CPE reduction assays were performed in monolayers of human foreskin fibroblast (HFF) cells (5000/well) in 384-well plates in assay media consisting of MEM with Earle's salts, 2% FBS and standard concentrations of L-glutamine, penicillin and gentamycin. 5000 cells were seeded in 384-well microtiter plates and incubated at 37° C. in a humidified 5% CO₂ incubator for 24 hours to allow for formation of confluent monolayers. Dilutions of antiviral test compounds, including filociclovir, were prepared directly in the plates in a series of 5-fold dilutions in duplicate wells to yield final concentrations that ranged from 300 to 0.1 μM or from 10 to 0.003 μM which allows for a large dynamic range of compound concentrations to facilitate the detection of antiviral activity of unknown compounds with either weak or potent antiviral activity.

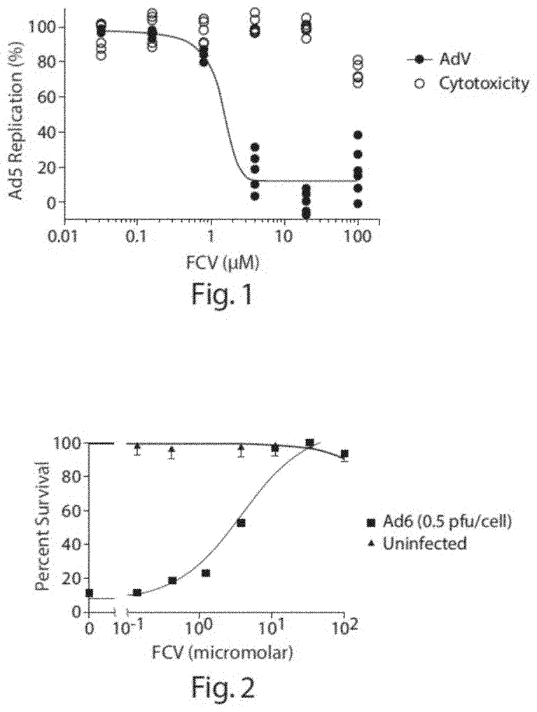

Cell monolayers were infected at a multiplicity of infection (MOI) of approximately 0.005 PFU/cell with virus strains including AdV5. Infected cells were incubated at 37° C. until 100% CPE was observed in the virus control wells. Cytopathology was determined by the addition of CellTiter-Glo® reagent (Promega, Madison, Wis.). Concentrations of antiviral test compounds sufficient to reduce CPE by 50% (EC₅₀) were interpolated from the experimental data using standard methods in Microsoft® Excel. Cytotoxicity was also determined with CellTiter-Glo® luminescent cell viability assay (Promega, Madison, Wis.) and concentrations of the test compounds that decreased cell viability by 50% (CC₅₀) were calculated from the data and selective index (SI) values were calculated as the CC₅₀/EC₅₀ as a measure of antiviral activity. The results are shown in Table 1 below and FIG. 1.

TABLE 1
Cytotoxicity of various antiviral compounds against Adenovirus 5

AdV 384 Well CPE^{a}

| Drug | EC₅₀ | CC₅₀ |
|---|---|---|
| CDV | 4.0 ± 2.0 | 97 ± 4 |
| PFA | >1000 ± 0 | >1000 ± 0 |
| GCV | 66 ± 15.1 | >100 ± 0 |
| ACV | >100 ± 0 | >100 ± 0 |
| PCV | >100 ± 0 | >100 ± 0 |
| FIAU | >100 ± 0 | >100 ± 0 |
| IDU | >100 ± 0 | >100 ± 0 |
| BDCRB | 70 ± 39 | 91 ± 13 |
| CMX001 | <0.03 ± 0 | 0.83 ± 0.7 |
| AZT | >100 ± 0 | >100 ± 0 |
| FCV | 2.2 ± 0.4 | 95 ± 10.6 |
| 4-thio-IDU | 11 ± 9.4 | 26 ± 24 |
| N-MCT | 57 ± 47 | 72 ± 33 |
| L-BHDU | >100 ± 0 | >100 ± 0 |
| PMEA | 48 ± 8 | >100 ± 0 |

^{a}Values shown represent the average EC₅₀ and CC₅₀ values (μM) and SD from five independent experiments.
Abbreviations:
CDX—cidofovir;
PFA—foscarnet;
GCV—ganciclovir;
ACV—acyclovir;
PCV—penciclovir;
FIAU—fluoroiodoarabinosyl adenine;
IDU—idoxuridine;
BDCRB—bromodichlororibobenzimidazole;
CMX001—brincidofovir;
AZT—azidothymidine;
FCV—filociclovir;
4-thio-IDU—4-thioidoxuridine;
N-MCT—N-methanocarbathymidine;
L-BHDU—L-Bromovinyl-Hydroxymethyl-Dioxolan Uracil;
PMEA—adefovir

The results shown in Table 1 demonstrate that filociclovir (FCV) exhibits significantly improved inhibitory activity against adenovirus 5 (SI=43) over all the other test compounds and was at least twice as potent as the next most effective antiviral agent, cidofovir (CDV) (SI=24) and significantly better than ganciclovir (GCV) (SI≥1.5).

The percent reduction in AdV5 replication as a function of filociclovir concentration ranging from 0.01 to 100 μM is shown in FIG. 1. The results in FIG. 1 demonstrate that concentrations of filociclovir as low as 0.2 μM are effective to reduce AdV replication.

Similar experiments were carried out with filociclovir against adenovirus 6, adenovirus 7, and adenovirus 8. The results are shown below in Table 2. Filociclovir exhibited potent inhibition of adenovirus 8 (SI≥61).

TABLE 2
Cytotoxicity of Filociclovir

| Virus | EC₅₀ | CC₅₀ | CC50/EC50 |
|---|---|---|---|
| AdV6 | 3.67 | >100 | 27 |
| AdV8 | 2.45 | >150 | 61 |
| AdV7 | 1.04 | >150 | >144 |

### Example 2. A Neutral Red Survival Assay to Measure Inhibition of Adenovirus 6 (AdV6) by Filociclovir

The neutral red cell cytotoxicity assay is used to detect cell viability or drug cytotoxicity. The principle of this assay is based on the detection of viable cells via the uptake of the dye neutral red. Neutral red is a eurhodin dye that stains lysosomes in viable cells. Viable cells can take up neutral red via active transport and incorporate the dye into their lysosomes, but non-viable cells cannot not take up this chromophore. Consequently, after washing, viable cells can release the incorporated dye under acidified-extracted conditions. The amount of released dye can be used to determine the total number of viable cells or drug cytotoxicity. As such, cytotoxicity is expressed as a concentration-dependent reduction of the uptake of neutral red after exposure to the compound under investigation.

Neutral red survival assays were carried out in a 96-well format. Briefly, A549 adenocarcinomic human alveolar basal epithelial cells were plated at a concentration of 6×10³ cells per well one day prior to infection. AdV6 infections were carried out at 5×10³ PFU per well (an estimated 0.5 PFU/cell at the time of infection). Filociclovir was serially diluted 1:3 on separate 96-well plates with a no drug final row as control. The AdV6 infections were performed as 9 replicate wells for each drug concentration and there were 3 uninfected drug control wells for each drug concentration. Filociclovir dilutions were added to the cell plates immediately before infection with AdV6. At 6 days post-infection (when viral cytopathic effect had reached 70-90% for the virus-infected, no drug wells), neutral red was added for 1 hour, then plates were washed 3 times with PBS to remove unattached cells, and the neutral red was extracted from the remaining cells using 50% ethanol/1% glacial acetic acid. Filociclovir plates were read and the results were graphed using GraphPad Prism graphing and statistical software (GraphPad Software, San Diego, Calif.).

The results are shown in FIG. 2 and Table 2. As seen in FIG. 2, filociclovir concentrations as low as 10⁻¹ μM were effective to improve viability of AdV6 infected cells.

### Example 3. Immunofluorescence Assay of Filociclovir-Treated AdV5 or AdV6 Infections of Human A549 Cells

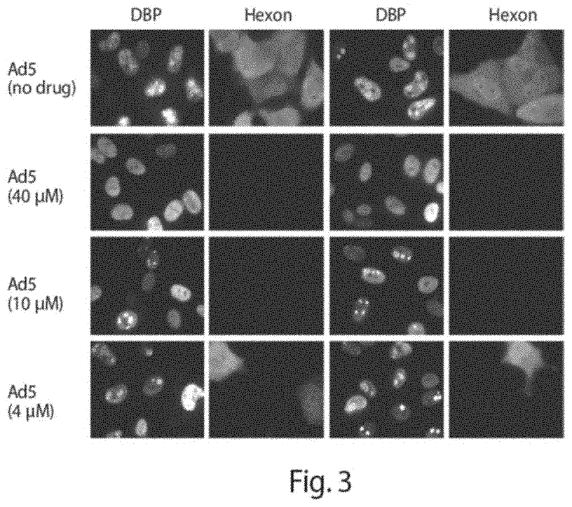

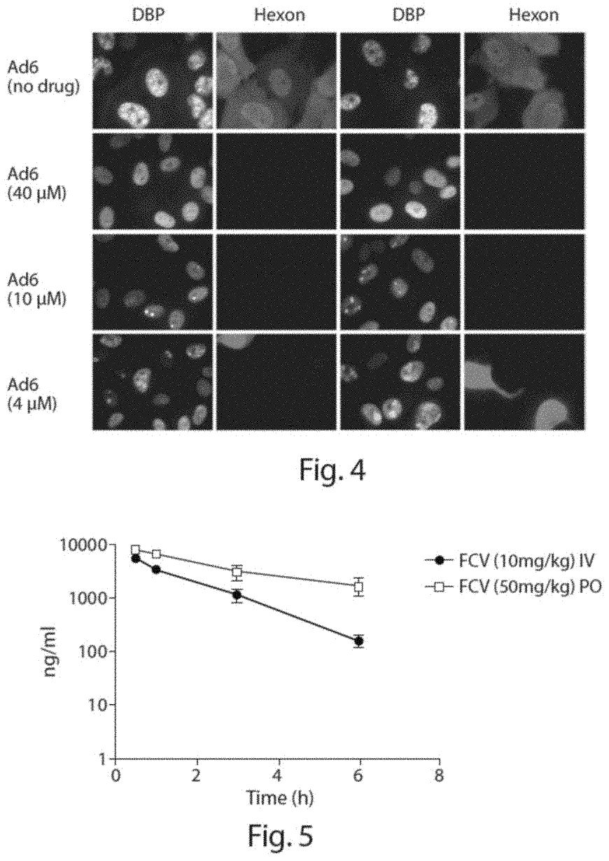

Human A549 cells on glass coverslips in 6-well plates were infected at an MOI of 5 PFU/cell with either AdV5 or AdV6 or were mock-infected. After 1 hour, filociclovir was added to a final concentration of 40, 10, 4, or 0 μM. At 27 hours post-infection, the A549 cells were fixed in paraformaldehyde (3.7% in PBS) and permeabilized with methanol. Cells were stained for the Adenovirus DNA-binding protein (DBP; green staining in images) and adenovirus hexon (red staining in images). DBP staining will be antinuclear and uniform during early AdV infection (prior to AdV DNA replication). DBP associates with replication centers as the infection progresses. The replication centers are initially small “dots” (each dot results from one incoming AdV genome). The replication centers will expand and “multiply” as DNA replication takes place. The nuclei become enlarged and misshapen as infection progresses. AdV hexon, the most abundant component of the AdV viral capsid, is not expressed until after DNA replication is taking place and is therefore considered to be a “late” AdV protein. The results are shown in FIGS. 3 and 4.

As seen in FIGS. 3 and 4, the results of filociclovir treatment was the same for AdV5 and AdV6 infection. Filociclovir was very effective in preventing late infection (as would be indicated by hexon staining). No hexon-positive cells were seen for the 40 μM filociclovir wells and extremely few hexon-positive cells for the 10 μM filociclovir wells. Some hexon-positive cells are seen with the 4 μM filociclovir wells, but the 4 μM wells show substantially less advanced infection (as indicated by the DBP staining patterns and the size/shape of nuclei) in comparison to the control (no drug) wells.

The results show that filociclovir is a potent inhibitor of human adenovirus 5 (AdV5), AdV6 and AdV8. This level of activity is similar to that of cidofovir (CDV) and far superior to that of ganciclovir (GCV), which has an IC₅₀ value of 66 μM against AdV5. (See, Table 1) The range of AdV serotypes tested so far suggest that FCV could potentially be developed as a pan-adenoviral inhibitor.

### Example 4. Pharmacokinetics of Filociclovir

In order to test the pharmacokinetics of filociclovir, Syrian hamsters were given either an oral (p.o. 50 mg/kg) or intravenous (i.v. 10 mg/kg) dose of filociclovir. The results are shown in FIG. 5 and demonstrate that filociclovir was still detectable 6 hours after PO or IV administration.

### Syrian Hamster AdV Model

The hamster model is particularly advantageous as it reproduces the pathology seen with human patients, and it can be used to test the efficacy of antiviral compounds (reviewed in Wold, W. S. M. and Toth, K., Advances in Cancer Research, 115: 69-92 (2012)). Syrian hamsters are one of the two rodent species (the other is the cotton rat) that are permissive to AdV species C infections (types 1, 2, 5, 6). Using this model, it is possible to carry out controlled in vivo experiments to test the efficacy of anti-adenoviral compounds (Ying et al., Antimicrobial Agents and Chemotherapy, 58(12): 7171-7181 (2014); Toth et al., Proc. Natl. Acad. Sci. USA, 105(20): 7293-7297 (2008); Tollefson et al. 2014; Toth et al., Viruses, 7(3): 1409-1428 (2015)). In these experiments, young hamsters were immunosuppressed with cyclophosphamide (CP), an agent often used as part of the conditioning regimen for human transplant recipients. After the desired degree of immunosuppression was achieved, the hamsters were infected intravenously (iv) with AdV5, leading to replication of AdV5 in most organs, most prominently in the liver (Toth et al. 2008 supra).

### Drug Formulation

For p.o. administration, filociclovir was suspended in 0.4% carboxymethyl cellulose (Sigma C5678) at 5 mg/ml and sonicated to visual homogeneity. For i.v. administration, filociclovir was dissolved in DMSO (Sigma D2650) and then diluted with water to a final concentration of 5 mg/ml for filociclovir and 75% DMSO. The dosing solutions were prepared one day before use and stored at 4° C.

### Experimental Design

Male hamsters were purchased from Envigo (Harlan) at ˜100 g body weight. All hamsters were immunosuppressed using cyclophosphamide (CP) administered intraperitoneally at a dose of 140 mg/kg, and then twice weekly at a dose of 100 mg/kg. The animals received three injections of CP before the administration of filociclovir. Two groups of animals (12 hamsters/group) were used for the two routes of administrations. In addition, three untreated animals (the same three hamsters for both routes) served as mock controls. The p.o. and i.v. experiments were performed on two consecutive days. As the hamsters weighed approximately 100 g, (+/−10%), they were administered 1 ml and 0.2 ml for the p.o. and i.v. routes, respectively, to achieve the desired dose. For both routes, 3 hamsters were sacrificed at 0.5, 1, 3, and 6 h post dosing.

Plasma and liver samples were collected from all animals and stored at −80° C. until analyzed by LC MS as follows:
All samples and standards were processed on ice;
Standard curve is constructed with authentic compound (fresh powder DMSO reconstitution spiked into “blank” Hamster plasma) and all samples spiked with 500 ng/mL carbamezapine (Internal Standard);
Early time point samples are diluted in “blank” hamster plasma (try to attain concentrations on the standard curve);
Sample extracted with 2× sample volume with 0.1% ammonium formate in methanol (i.e. 30 μL sample+60 μL 0.1% ammonium formate in methanol);
Extracted samples centrifuged on maximum setting in microfuge;
Supernatants are analyzed by LC/MS/MS Dynamic Multiple Reaction Monitoring (DMRM) utilizing custom method.

### Example 5. Determining the Therapeutic Efficacy of Filociclovir Against Intravenously Administered Human AdV6 in Immunosuppressed Male Syrian Hamsters

As set forth above, it has been established that filociclovir is an effective inhibitor against several strains of adenovirus replication in vitro and demonstrates good oral bioavailability in hamsters.

Next, we assessed whether filociclovir exhibits anti-adenoviral efficacy in immunosuppressed Syrian hamsters infected intravenously (i.v.) with AdV6. Four dose levels were tested: 10, 30, 60 and 100 mg/kg p.o. q.d. The highest dose was determined based on rat toxicology data.

### Experimental Design

All hamsters were immunosuppressed using cyclophosphamide (CP). CP was administered intraperitoneally at a dose of 140 mg/kg, and then twice weekly at a dose of 100 mg/kg.

Filociclovir, in powdered form, was suspended in 0.4% carboxymethyl cellulose (Sigma C5678, Lot #SLBS7273) at 1, 3, 6, and 10 mg/ml and sonicated to visual homogeneity and aliquots stored at 4° C. Aliquots were allowed to equilibrate to room temperature before dosing. Hamsters weighing approximately 100 g were dosed with a 1 ml volume of the appropriate suspension for the 10, 30, 60, and 100 mg/kg dose levels.

The hamsters were divided into 8 groups, 15 hamsters/group, (with the exception of Group 2, which had only 5 hamsters; see Table 3), immunosuppressed, and then injected i.v. with vehicle (Groups 1-2) or 2×10¹⁰ PFU/kg of AdV6 (Groups 5-9). Groups 1 and 3 received drug vehicle (p.o. q.d.), Group 4 received 10 mg/kg filociclovir (p.o. q.d.), Group 5 received 30 mg/kg filociclovir (p.o. q.d.), Group 6 received 60 mg/kg filociclovir (p.o. q.d.), Groups 2 and 7 received 100 mg/kg filociclovir (p.o. q.d.), and Group 8 received cidofovir (20 mg/kg, 3 times weekly). For all groups, drug administration started 1 day before challenge, and continued according to the schedule above for the duration of the study.

The body weights and any signs of morbidity of the animals were recorded daily. At 5 days post challenge, 5 hamsters (designated at the start of the experiment) from each group (with the exception of Group 2) were sacrificed, and gross pathological observation was performed. Serum and liver samples were collected and the virus burden in liver was determined by a TCID50 assay, and the serum was analyzed for transaminase levels. The remaining 10 hamsters were sacrificed at 14 days post challenge. Hamsters that became visually moribund before Day 14 were sacrificed as needed. In addition to the hamsters observed to be moribund, all hamsters that lost more than 20% of their original body weight were also sacrificed. For the hamsters sacrificed at the conclusion of the experiment and those sacrificed as moribund, serum and liver tissues were collected and banked for possible determination of serum transaminase levels and virus burden in the liver. Liver tissue was preserved in formalin for possible histopathological examination.

As such, there were two endpoints for the study: One, collected from ten animals, was survival and body weight gain/loss (with the option of assaying virus burden in the liver and serum transaminase levels). The other, collected from 5 animals for the Day 5 time point, was virus burden in the liver and serum transaminase levels.

TABLE 3
Therapeutic window study for Filociclovir - AdV6-infected male animals.

| | | | Work Performed | | |
|---|---|---|---|---|---|
| | | | | At Necropsy | |
| Group # | Groups | n | Daily | Day 5 | Day 14 |
| 1 | Vehicle | 5 + 10 | Dose drug/drug | Serum ALT, | Bank serum for |
| | | | vehicle daily, | bank liver for | ALT, liver for |
| | | | Observation, | histopathology | histopathology |
| 2¹ | Vehicle + | 5 | Body Weight | | Bank serum for |
| | filociclovir | | | | ALT, liver for |
| | 100 mg/kg | | | | histopathology |
| | p.o. q.d. | | | | |
| 3 | AdV6 + | 5 + 10 | | Serum ALT, | Bank serum for |
| | Vehicle | | | liver TCID50, | ALT, liver for |
| 4 | AdV6 + | 5 + 10 | | bank liver for | TCID50, liver for |
| | filociclovir | | | histopathology | histopathology |
| | 10 mg/kg | | | | |
| | p.o. q.d. | | | | |
| 5 | AdV6 + | 5 + 10 | | | |
| | filociclovir | | | | |
| | 30 mg/kg | | | | |
| | p.o. q.d. | | | | |
| 6¹ | AdV6 + | 5 + 10 | | | |
| | filociclovir | | | | |
| | 60 mg/kg | | | | |
| | p.o. q.d. | | | | |
| 7¹ | AdV6 + | 5 + 10 | | | |
| | filociclovir | | | | |
| | 100 mg/kg | | | | |
| | p.o. q.d. | | | | |
| 8 | AdV6 + | 5 + 10 | | | |
| | CDV | | | | |
| | 20 mg/kg | | | | |
| | i.p. 3x weekly | | | | |

¹Removed from study

### Results

### In-Life Observations

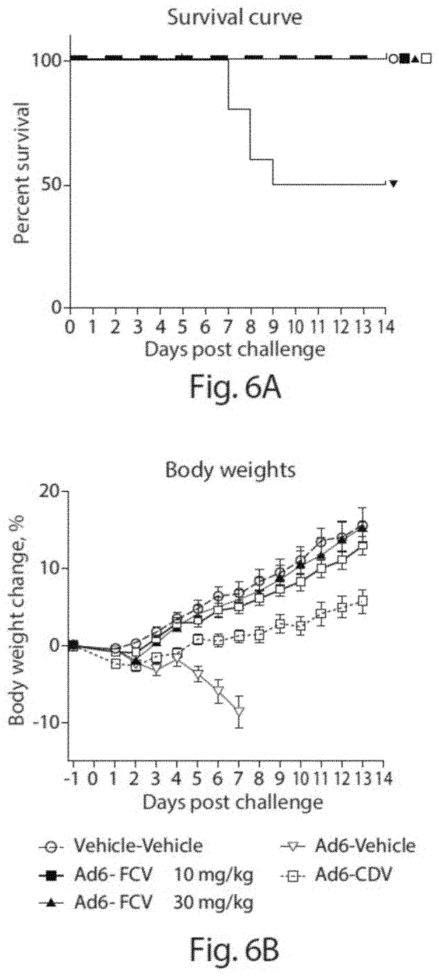

There were 5 treatment-related deaths in the experiment, all in the AdV6+Vehicle group (FIG. 6A). Filociclovir at 10 or 30 mg/kg prevented mortality (FIG. 6A). Initially, all animals in the AdV6-infected groups lost body weight; this was reversed by treatment with 10 mg/kg or 30 mg/kg filociclovir (FIG. 6B). The weight gain of animals in the AdV6+10 mg/kg filociclovir was slightly lower than that of uninfected animals and animals in the AdV6+30 mg/kg filociclovir group. Higher doses of filociclovir, especially the 100 mg/kg dose, are toxic to AdV6-infected animals (not shown); these groups were removed from the study.

### Necropsy

At the day 5 (D5) sacrifice, extensive kidney pathology was observed for animals in the AdV6+60 mg/kg and 100 mg/kg filociclovir groups.

### Serum Transaminase Levels

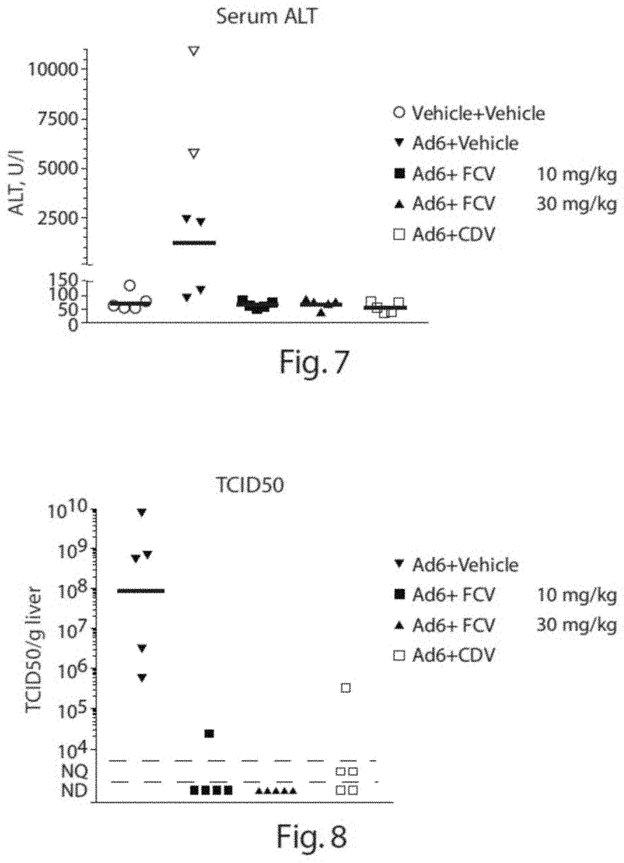

At 5 days post challenge, serum was collected from 5 animals in each group and analyzed for transaminase levels. Two hamsters in the AdV6+Vehicle group had high transaminase levels, while none of the filociclovir- or CDV-treated animals had elevated serum transaminase levels (FIG. 7; alanine aminotransferase [ALT] shown).

### Virus Burden in the Liver

At 5 days post challenge, untreated AdV6-infected hamsters had high virus burden in their liver (FIG. 8). Treatment with 30 mg/kg filociclovir suppressed AdV6 replication to a nondetectable level (FIG. 8). There was one animal in the AdV6+10 mg/kg filociclovir group with a very low liver virus burden, while we could not detect AdV6 in the liver of any of the other animals in this group (FIG. 8). CDV treatment reduced virus replication to very low to nonquantifiable levels (FIG. 8).

### CONCLUSIONS

Data with the two lower doses of filociclovir (10 mg/kg and 30 mg/kg) are promising. Treatment with either of these two doses inhibited virus replication (FIG. 8), mitigated liver damage (FIG. 7), and prevented morbidity and mortality (FIG. 6). The 10 mg/kg filociclovir dose is slightly less efficacious than the 30 mg/kg dose (see marginally lower body weight gain in FIG. 6 and marginally higher virus burden in the liver in FIG. 8). The 60 mg/kg and 100 mg/kg filociclovir doses are toxic for AdV6-infected hamsters; these two groups and the Vehicle+100 mg/kg group were removed from the study.

### Example 6. In Vitro Inhibition of Ocular-Associated Adenovirus Strains by Filociclovir

The effectiveness of filociclovir to inhibit a number of adenovirus strains associated with conjunctivitis were tested in an in vitro assay. To determine the in vitro IC₅₀ concentrations, i.e., the concentration of filociclovir that inhibits adenovirus plaque formation by 50%, filociclovir and cidofovir (CDV) were tested against a panel of 7 ocular types of adenovirus.

### Plaque Reduction Antiviral IC₅₀ Assay

42 A549 24-well multiplates (1 per each drug and each virus strain) were prepared. 7 adenovirus serotypes were tested at a time. Virus dilutions from known titer of stock viruses (approx. 100 PFU/0.1 ml) were prepared with AdV3 L, AdV4 H, AdV5 M, AdV7a J, AdV8 E, AdV 19/64 K, and AdV37 ATCC. 0.1 ml (containing approx. 100 PFU) of each of the 7 viruses were plated onto all 24 wells of an A549 multiplate and incubated at 37° C. in a 5% CO₂ water-vapor atmosphere for 3 hours.

Dilutions of filociclovir and cidofovir were prepared at 400, 40, 4.0, 0.4, 0.04, and 0.004 μM in 2× nutrient tissue culture media, following the dilution protocol: 7.5 ml of each of filociclovir and cidofovir dilution was added to a tube and 22.5 ml of methylcellulose in outgrowth medium (OG) was then added to each tube. 1 ml of each filociclovir and cidofovir dilutions was added to an additional 15 ml tube and 3 ml of OG was added to each tube. This was the overlay for the Adv8 plates. Normal methylcellulose overlay medium without any drug was used for the control wells for each plate.

For each virus plate, 3 wells were overlaid with each of the 6 final filociclovir and cidofovir concentrations (100, 10, 1.0, 0.1, 0.01, and 0.001 μM) and 6 wells with the control media. Plates were incubated at 37° C. in a 5% CO₂ water-vapor atmosphere until plaques were visible. When plaques became visible, plates were stained with gentian violet and plaques were counted at 25λ using a dissecting microscope. The IC₅₀ concentrations were determined from Fitted Line Plot regression analysis using Minitab. All assays were performed in triplicate.

### Results

Filociclovir produced mean IC₅₀ concentrations of <5 μM (range 0.496-4.684 μM) across a panel of ocular adenovirus types (Ad3, Ad4, Ad5, Ad7a, Ad8, Ad19/64, and Ad37) representing viral types for adenovirus Species B, C, D, and E. Cidofovir produced mean IC₅₀ concentrations of <31 μM (range 0.487-30.304 μM) across the same panel of ocular adenovirus types and species. The mean and median IC₅₀ (μM) for filociclovir and cidofovir are shown in Tables 4 and 5, respectively.

TABLE 4
Mean IC₅₀ [μM] for Filociclovir and Cidofovir

| | Mean IC₅₀ | Mean IC₅₀ |
|---|---|---|
| Virus | Filociclovir | Cidofovir |
| AdV3 | 0.778 | 5.782 |
| AdV4 | 4.313 | 8.708 |
| AdV5 | 4.684 | 30.304 |
| AdV7a | 2.119 | 1.808 |
| AdV8 | 0.496 | 0.487 |
| AdV37 | 3.533 | 3.960 |
| AdV64 | 1.856 | 4.092 |

TABLE 5
Median IC₅₀ [μM] for Filociclovir and Cidofovir

| | Median IC₅₀ | Median IC₅₀ |
|---|---|---|
| Virus | Filociclovir | Cidofovir |
| AdV3 | 0.756 | 6.020 |
| AdV4 | 4.236 | 9.429 |
| AdV5 | 4.625 | 38.788 |
| AdV7a | 0.769 | 0.617 |
| AdV8 | 0.485 | 0.474 |
| AdV37 | 4.516 | 0.382 |
| AdV64 | 0.424 | 4.937 |

### Conclusion

Filociclovir proved to be an effective inhibitor of all ocular-associated adenovirus strains tested. In many cases, filociclovir proved to be a significantly more potent adenovirus inhibitor than cidofovir. This is a strong indication that filociclovir is a safe and effective treatment for adenovirus-associated conjunctivitis.

### Example 7. In Vivo Analysis of Filociclovir in a Rabbit Ocular Adenovirus Model

The ability of filociclovir to inhibit adenovirus-related conjunctivitis was tested in a New Zealand White (NZW) rabbit ocular model.

20 NZW rabbits (1.1-1.4 kg) were given general anesthesia with ketamine & xylazine, topical anesthesia with proparacaine, and corneal scarification (12 cross-hatched strokes of a #25 needle) followed by inoculation in both eyes with 50 μl of 3.0×10⁷ PFU/ml of AdV5 M (6.0×10⁸ PFU/ml). Eyes were closed and gently rubbed for 5 seconds to ensure contact of the virus on all ocular surfaces. All rabbits were treated with analgesia in the form of intramuscular injections of ketoprofen (1.5 mg/kg).

Beginning 2 hours after inoculation, all eyes were cultured for virus. Following topical anesthesia with proparacaine, a single cotton-tipped swab was placed into the lower fornix of each eye, rolled over the cornea into the upper fornix to recover adenovirus from the tear film and corneal and conjunctival surfaces. The swabs from each eye were placed individually into tubes containing 1 ml of outgrowth media and were frozen at −80° C. pending plaque assay.

On Day 1, the rabbits were divided into 4 treatment groups for drug administration as set forth in the chart below. The four treatment groups were 0.5% Filociclovir; 0.1% Filociclovir; 0.5% cidofovir; and the negative vehicle control (10% 2-Hydroxpropyl)-β-cyclodextrin, 0.2% cremophore).

| | | Treatment | n | n | Rabbit |
|---|---|---|---|---|---|
| Group | Drug | Regimen | Rabbits | Eyes | Numbers |
| 1A | 0.5% Filociclovir | 4 times daily | 5 | 10 | 1-5 |
| | | for 10 days | | | |
| 1B | 0.1% Filociclovir | 4 times daily | 5 | 10 | 6-10 |
| | | for 10 days | | | |
| 1C | 0.5% Cidofovir | 2 times daily | 5 | 10 | 11-15 |
| | | for 7 days | | | |
| 1D | Vehicle Control | 4 times daily | 5 | 10 | 16-20 |
| | | for 10 days | | | |

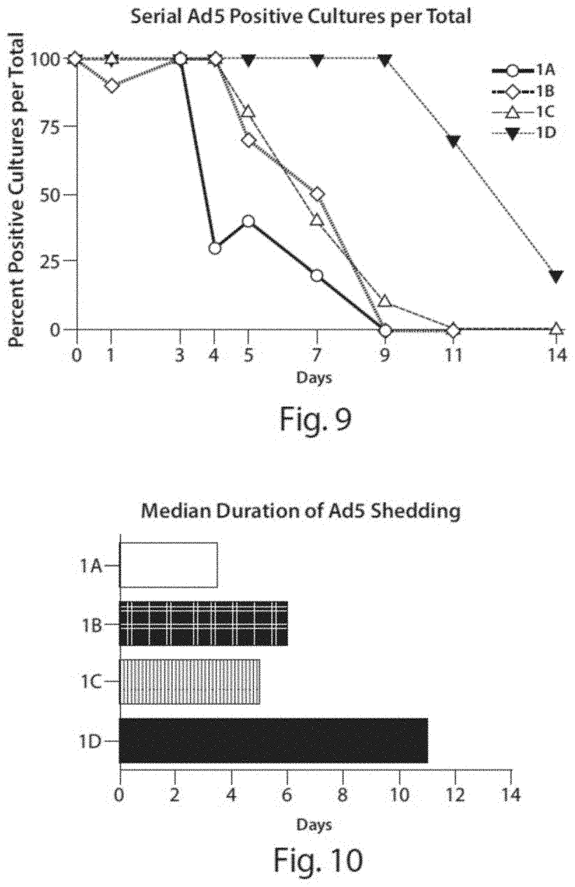

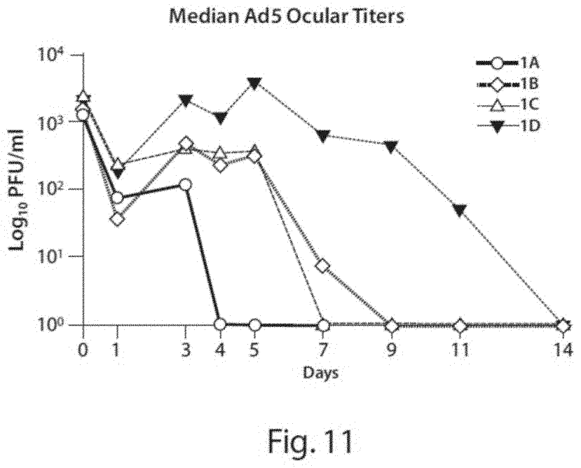

Treatments began on Day 1 and drops were administered with at least a 2-hour interval between drops. The eyes of each rabbit were cultured for virus on days 1, 3, 4, 5, 7, 9, 11, and 14 post-inoculation (PI) at least 1 hour after the final drop. At various times during the course of the experiment, AdV5 titers were determined on A549 cell monolayers using a standard plaque assay. The ocular cultures to be titered were thawed, diluted (1:10) and inoculated onto A549 monolayers. The virus was adsorbed for 3 hours. Following adsorption, 1 ml of media plus 0.5% methylcellulose was added to each well, and the plates were incubated at 37° C. in a 5% CO₂-water vapor atmosphere. After 7 days incubation, the cells were stained with 0.5% gentian violet, and the numbers of plaques were counted under a dissecting microscope (25λ). The viral titers were then calculated and expressed as plaque-forming units per milliliter (PFU/ml). The data were analyzed statistically using True Epistat and/or Minitab statistical software. Outcome measures included Daily AdV5-Positive Cultures per Total (FIG. 9), Duration of Shedding (FIG. 10) and Daily Viral Titers (FIG. 11). Significance was established at the p≤0.05 confidence level.

### Results

Treatment with 0.5% filociclovir demonstrated significantly better antiviral efficacy compared with the Vehicle control. Specifically, filociclovir demonstrated a fewer number of adenovirus positive cultures per total on Days 4, 5, 7, 9, and 11 (see, FIG. 9), a shorter duration of adenovirus shedding (see, FIG. 10), significantly lower adenovirus titers on Days 1, 3, 4, 5, 7, 9, and 11 (see, FIG. 11). Treatment with 0.5% filociclovir also demonstrated significantly better antiviral efficacy compared with the positive antiviral control, 0.5% cidofovir, with filociclovir showing significantly lower adenovirus titers on Days 1, 3, 4, and 5, and a fewer number of adenovirus positive cultures per total on Day 4.

Treatment with 0.5% filociclovir also demonstrated significantly better antiviral efficacy compared with the 0.1% filociclovir group, with 0.5% filociclovir exhibiting significantly lower adenovirus titers on Days 3, 4, and 5 (see, FIG. 11), and fewer number of adenovirus positive cultures per total on Day 4 (see, FIG. 9).

Treatment with 0.1% filociclovir did demonstrate considerably better antiviral efficacy compared with the Vehicle control, with significantly lower adenovirus titers on Days 1, 3, 4, 5, 7, 9, and 11 (see, FIG. 11), fewer number of adenovirus positive cultures per total on Days 7, 9, and 11 (see, FIG. 9) and a shorter duration of shedding (see, FIG. 10).

Treatment with 0.1% filociclovir also demonstrated similar antiviral efficacy compared with the positive antiviral control, 0.5% cidofovir, except for Day 1 where the 0.1% filociclovir titers were lower.

Treatment with 0.5% cidofovir demonstrated significantly better antiviral efficacy than the Vehicle control, specifically 0.5% cidofovir exhibited significantly lower adenovirus titers on Days 3, 4, 5, 7, 9, and 11 (see, FIG. 11), a fewer number of adenovirus positive cultures per total on Days 7, 9, and 11 (see, FIG. 9) and a shorter duration of shedding (see, FIG. 10).

### Conclusions

0.5% and 0.1% filociclovir demonstrated significant antiviral efficacy compared with the Vehicle Control in the adenovirus/NZW rabbit ocular model. 0.5% filociclovir was more effective than the positive antiviral control, i.e., 0.5% cidofovir, during the early phase of infection (Days 1-5). 0.1% Filociclovir demonstrated similar antiviral efficacy as the positive antiviral control, 0.5% cidofovir, in the adenovirus/NZW rabbit ocular model.

0.5% cidofovir demonstrated significant antiviral efficacy in the adenovirus/NZW rabbit ocular model, thus validating the study.

Therefore, the data presented above from the standard in vivo adenovirus/NZW rabbit ocular model demonstrate that filociclovir provides a safe and effective method for the treatment and prevention of ocular diseases associated with adenoviral infections of the eye.

Consideration of the foregoing data indicates that filociclovir is a safe and effective treatment for conjunctivitis as well as demonstrating that filociclovir is an effective antiviral agent for inhibiting a range of adenovirus types, leading to the conclusion that filociclovir is useful as a pan-adenoviral therapeutic for viral infections of the eye.

All publications, patent applications, patents, and other documents cited herein are incorporated by reference in their entirety. The examples set forth above are illustrative only and not intended to be limiting. Obvious variations to the disclosed methods and alternative embodiments of the invention will be apparent to those skilled in the art in view of the foregoing disclosure. All such obvious variants and alternatives are considered to be within the scope of the invention as described herein

What is claimed is:

1. A method of treating or preventing a viral infection of the eye in a mammal comprising topically administering to said eye an effective amount of a composition comprising filociclovir or a pharmaceutically acceptable salt thereof.

2. The method according to claim 1, wherein said viral infection is caused by cytomegalovirus (CMV), Epstein-Barr virus (EBV), varicella zoster virus (VZV), adenovirus (AdV), HHV-6A virus, HHV-6B virus, HHV-8 virus, JC virus, and BK virus, or a combination thereof.

3. The method according to claim 1, wherein said viral infection is caused by adenovirus.

4. The method according to claim 3, wherein said adenovirus is selected from the group consisting of adenovirus 1, 2, 3, 4, 5, 6, 7, 7a, 8, 9, 10, 11, 13, 14, 15, 16, 17, 20, 21, 22, 23, 24, 25, 26, 27, 28, 29, 30, 32, 33, 36, 37, 38, 39, 42, 43, 44, 45, 46, 47, 48, 49, 53, 54, 56, and 64.

5. The method according to claim 4, wherein said adenovirus is selected from the group consisting of adenovirus 3, 4, 5, 6, 7, 7a, 8, 37, 54, and 64.

6. The method according to claim 4, wherein said adenovirus is adenovirus 3.

7. The method according to claim 4, wherein said adenovirus is adenovirus 4.

8. The method according to claim 4, wherein said adenovirus is adenovirus 5.

9. The method according to claim 4, wherein said adenovirus is adenovirus 6.

10. The method according to claim 4, wherein said adenovirus is adenovirus 7.

11. The method according to claim 4, wherein said adenovirus is adenovirus 7a.

12. The method according to claim 4, wherein said adenovirus is adenovirus 8.

13. The method according to claim 4, wherein said adenovirus is adenovirus 37.

14. The method according to claim 4, wherein said adenovirus is adenovirus 54.

15. The method according to claim 4, wherein said adenovirus is adenovirus 64.

16. The method according to claim 1, wherein the composition is topically administered to the cornea and/or conjunctiva of the eye.

17. The method according to claim 1, wherein said mammal is a human.

18. The use of filociclovir in the manufacture of a medicament for topical administration for use in a method for treating or preventing a viral infection of the eye in a mammal.

19. The use according to claim 18, wherein said viral infection is caused by cytomegalovirus (CMV), Epstein-Barr virus (EBV), varicella zoster virus (VZV), adenovirus (AdV), HHV-6A virus, HHV-6B virus, HHV-8 virus, JC virus, and BK virus, and a combination thereof.

20. The use according to claim 19, wherein said viral infection is caused by adenovirus.

21. The use according to claim 20, wherein said adenovirus is selected from the group consisting of adenovirus 1, 2, 3, 4, 5, 6, 7, 7a, 8, 9, 10, 11, 13, 14, 15, 16, 17, 20, 21, 22, 23, 24, 25, 26, 27, 28, 29, 30, 32, 33, 36, 37, 38, 39, 42, 43, 44, 45, 46, 47, 48, 49, 53, 54, 56, and 64.

22. The use according to claim 21, wherein said adenovirus is selected from the group consisting of adenovirus 3, 4, 5, 6, 7, 7a, 8, 37, 54, and 64.

23. The use according to claim 18, wherein the composition is topically administered to the cornea and/or conjunctiva of the eye.

24. The use according to claim 18, wherein said mammal is a human.

25. An ophthalmic composition comprising an ophthalmically acceptable carrier and filociclovir in an amount effective to treat a viral infection of the eye.

26. The composition of claim 25, wherein the composition comprises a topically ophthalmic acceptable carrier for administration to the eye.

27. The composition of claim 26, wherein said topically ophthalmic acceptable carrier comprises an aqueous solution, a non-aqueous solution, an oil, wax, grease, petrolatum, or a combination thereof.

28. The composition of claim 27, wherein the ophthalmic composition is selected from the group consisting of an aqueous solution, a non-aqueous solution, a suspension, a solution/suspension, a gel, a cream, an ointment, and an emulsion.

29. The composition of claim 25 further comprising at least one excipient selected from the group consisting of a stabilizer, a penetrating enhancer, a pH adjusting agent, an antimicrobial preservative, a lubricant, a viscosifier, and a wetting agent.

30. The composition of claim 25, further comprising carbomer, triethanolamine, paraben, propylene glycol, and/or glycerin.

31. The composition of claim 25, wherein the amount of the compound is in the range of about 0.001% to 30% by weight.

32. The composition of claim 25, wherein said composition is topically administered to the cornea and/or conjunctiva of the eye.
